SEQUENCE LISTING (17 sequences)

SEQ ID NO: 1 - DNA, 24 bp, Artificial Sequence
  resistance determinant
actaggtgttggtgaagatatacc

SEQ ID NO: 2 - DNA, 24 bp, Artificial Sequence
  resistance determinant
gaacatgaattacacgagggcaaa

SEQ ID NO: 3 - DNA, 22 bp, Artificial Sequence
  resistance determinant
cctggaaccatgagtgttattg

SEQ ID NO: 4 - DNA, 22 bp, Artificial Sequence
  resistance determinant
gaacgtcagagaggaattacaa

SEQ ID NO: 5 - DNA, 22 bp, Artificial Sequence
  resistance determinant
ttcaccccaggcataatggtta

SEQ ID NO: 6 - DNA, 25 bp, Artificial Sequence
  resistance determinant
tcaattcatcttcaaaccttttctt

SEQ ID NO: 7 - DNA, 21 bp, Artificial Sequence
  resistance determinant
agcccaatattttagttgggg

SEQ ID NO: 8 - DNA, 21 bp, Artificial Sequence
  resistance determinant
atccgtactgatgttataagg

SEQ ID NO: 9 - DNA, 21 bp, Artificial Sequence
  resistance determinant
tctgataagtgagctattcac

SEQ ID NO: 10 - DNA, 17 bp, Artificial Sequence
  resistance determinant
tggagactnctcagtgt

SEQ ID NO: 11 - DNA, 20 bp, Artificial Sequence
  control
cgaaggtgggacaaatgatt

SEQ ID NO: 12 - DNA, 18 bp, Artificial Sequence
  control
tctagacagccactcata

SEQ ID NO: 13 - DNA, 19 bp, Artificial Sequence
  control
gattggacgagtcaggagc

SEQ ID NO: 14 - DNA, 19 bp, Artificial Sequence
  control
gctcctgactcgtccaatc

SEQ ID NO: 15 - DNA, 22 bp, Artificial Sequence
  control
agttctgcagtaccggatttgc

SEQ ID NO: 16 - DNA, 23 bp, Artificial Sequence
  primer
gtgcattgccagatgttcgtgat

SEQ ID NO: 17 - DNA, 21 bp, Artificial Sequence
  primer
gcttaacttagcttcagtgta

(19) United States
(12) Patent Application Publication
(10) Pub. No.: US 2006/0210998 A1
(43) Pub. Date: Sep. 21, 2006

(54) Determination of antibiotic resistance in staphylococcus aureus

(76) Inventors: Christiane Kettlitz, Gross Quenstedt (DE); Birgit Stromenger, Wernigerode (DE); Guido Werner, Wernigerode (DE); Wolfgang Witte, Elend (DE)

Correspondence Address: BELL, BOYD & LLOYD, LLC, PO BOX 1135, CHICAGO, IL, 60690-1135, US

(21) Appl. No.: 11/083,787

(22) Filed: Mar. 18, 2005

(51) Int. Cl. C12Q 1/68; C12M 1/34

(52) U.S. Cl. 435/6; 435/287.2

(57) ABSTRACT

The present invention relates to the detection of antibiotic resistance determinants in Staphylococcus aureus. The present invention discloses a micro-array for the detection of antibiotic resistance determinants and mutations in said organism, a method for the detection of said determinants and mutations and a kit.

### FIELD OF THE INVENTION

The present invention relates in general to the detection of antibiotic resistance determinants and in particular to detection of antibiotic resistance determinants in Staphylococcus aureus (S. aureus). The present invention specifies a DNA micro-array for the detection of antibiotic resistance determinants and mutations in said organism, a method for the detection of said determinants and mutations and a kit. This micro-array concept offers the rapid sensitive and specific identification of antibiotic resistance profiles. It is easily expandable and thus can be adapted to change clinical and epidemiological requirements in clinical diagnosis as well as in epidemiological studies.

### BACKGROUND OF THE INVENTION

S. aureus is one of the most common causes of nosocomial infections worldwide with the prevalence of methicillin-resistant S. aureus (MRSA) hiving been increased constantly during the past 15 years in many areas of the world (Witte, W.; J. Antimicrob. Chemother. 44 Suppl A (1999) pp. 1-9). It has been shown that severe infections with methicillin- and multi-resistant S. aureus are associated with an increased rate of mortality as well as with prolonged hospitalization ensuing increased health care costs as compared to infections with susceptible isolates. One reason might be a delay in adequate treatment since conventional identification and susceptibility testing in clinical microbiology is a time consuming process. In addition, problems arise from the heterogeneous expression of some resistance genes in vitro [for example expression of methicillin resistance (Chambers, H. F.; Clin. Microbiol. Rev. 10 (1997) pp. 781-791)] leading to unreliable treatment recommendations. To overcome limitations of classical susceptibility testing, rapid molecular tests are required for the detection of resistance causing determinants (Fluit, A. C. et al.; Clin. Microbiol. Rev. 14 (2001) pp. 836-71; Sundsfjord, A. et al.; 2004APMIS 112 (2004) pp. 815-837).

In principle, nucleic acid sequences isolated from clinical samples may be analyzed by using either gel electrophoresis of DNA fragments (e.g. of restriction fragments)—the so-called southern blot, hybridization events, or the direct sequencing of DNA (for example according to the Maxam-Gilbert method). All of the above-mentioned methods are widely spread in biological sciences, medicine and agriculture. The deficiencies of the three methods lie in that even though southern blots and hybridization experiments may be carried out relatively fast, they are useful merely for the analysis of short DNA strands. The DNA sequencing results in the accurate determination of the nucleic acid sequences, but is time consuming, expensive and connected with certain efforts when applied to greater projects, e.g. the sequencing of a complete genome.

Known methods to detect the presence of S. aureus in a clinical sample rely for example on the detection of methicillin-resistant S. aureus via annealing of specific probes (cf. US2005019893). Other approaches base on the use of medium for the specific detection of said strain (cf. US2004121404) and PCR methods employing for example primers deduced from the internal transcribed spacer region, which is located between the 16S and 23S ribosomal ribonucleic acid (rRNA) or rRNA genes (WO2004052606).

In contrast to PCR methods, micro-array technology provides a tool for a highly specific parallel detection of thousands of different DNA sequences in a single experiment (Schena, M. et al.; Science 270 (1995), 467-470). Micro-arrays which are in some cases also referred to as hybridization arrays, gene arrays or gene chips comprise in brief a carrier or support on which at defined locations at a possibly high density capture molecules are attached directly or via a suitable spacer molecule. The spacer molecules may be considered to function as a “bridge” between the capture molecule and the surface of the carrier to allow an easier attachment of the capture molecule. Said capture molecules consist of relatively short nucleic acid sequences, in particular DNA, which is capable to hybridize specific to the target molecules or probe molecules to be analyzed resulting usually in DNA:DNA or DNA:RNA hybrids. The occurrence of the hybridization event is then determined with for example fluorescent dyes and analyzed.

The advantages of the micro-array concept preliminary resides in its ability to carry out very large numbers of hybridization-based analyses simultaneously. Methods for the preparation of micro-arrays are exemplified in Maniatis et al., Molecular Cloning—A Laboratory Manual, First Edition, Cold Spring Harbor, 1982.

Originally developed for the analysis of mammalian gene expression, an increasing number of reports on micro-arrays for identification and characterization of prokaryotes also used in microbial diagnostics was encountered in recent years (Bodrossy, L. and A. Sessitsch; Curr. Opin. Microbiol. 7 (2004), 245-254). Combination of PCR based pre-amplification steps with subsequent micro-array based detection of amplicons on a micro-array facilitates the sensitive and highly specific detection of PCR products (Call, D. R. et al.; Int. J. Food Microbiol. 67 (2001), 71-80). Amplicons are identified by a specific hybridization reaction on the array thus reducing the risk of wrong positive results due to the occurrence of nonspecific bands after PCR. Besides that, micro-arrays utilizing oligonucleotides as capture probes enable the detection of single nucleotide polymorphisms (SNPs) such as resistance mutations without the need for additional sequencing. However, only a few studies describe the development of diagnostic micro-arrays for the molecular detection of bacterial antibiotic resistance, targeting either a limited number of acquired antibiotic resistance genes or resistance mutations in various genes.

The WO 01/7737 relates to the identification of (micro-)organisms among others having homologous nucleotide sequences via identification of their nucleotide sequences, after amplification by a single primer pair. Organisms of the same genus or family and/or related genes in a specific (micro) organism present in a biological sample may be identified or quantified.

In WO 03/031654 a micro-array with probes for genotyping Mycobacteria species, differentiating Mycobacterium strains and detecting antibiotic-resistant strains is disclosed. The simultaneous performance on multiple clinical isolates via a single test of a Mycobacterium genotyping test, M. tuberculosis strain differentiation test and an antibiotic-resistance detection test is specified.

Methods for assaying drug resistance and kits for performing such assays are disclosed in U.S. Pat. No. 6,013,435. Target sequences associated with genetic elements are selectively amplified and detected. The methods described are especially useful for screening micro-organisms, which are difficult to culture.

In U.S. Pat. No. 2,003,143591 methods and strategies to detect and/or quantify nucleic acid analytes in micro-array applications, such as genotyping (SNP analysis) are disclosed. In the methods referred to nucleic acid probes with covalently conjugated dyes are attached either to adjacent nucleotides or at the same nucleotide of the probe with the dyes being attached to the probes via novel linker molecules.

The state of the art still exhibits some disadvantages in that actually available methods for the determination of antibiotic resistant S. aureus species require long runs and are solely adaptive to a limited number of samples to be tested while also being expensive. Additionally, the present assays do not allow to achieve an overview on the resistance properties of a single strain and thus gives valuable and sometimes life-saving information about a suitable treatment.

### SUMMARY OF THE INVENTION

The present invention provides a micro-array, which incorporates nucleic acids for targeting at least 5 determinants and at least one resistance mutation of multi-resistant S. aureus, and thus enables a rapid, accurate and inexpensive identification of antibiotic resistance profiles. Said micro-array is easily expandable and may thus be adapted to changing clinical and epidemiological requirements in clinical diagnosis as well as in epidemiological studies. The present fast and reliable assay allowing a high throughput will be helpful in reducing the spread of multi-resistant isolates and will improve the treatment options of severe and sometimes life-threatening staphylococcal infections.

In the course of the extensive experimentation leading to the present invention various sequences have been investigated for their aptitude to cover a huge number of different resistant strains, while not exhibiting a substantial level of cross reactivity. It has been found that all of the strains investigated essentially contained at least one of the determinants and an endogenous resistant mutation.

The term “micro-array” as used herein refers to a carrier or support respectively, which is preferably solid and has a plurality of molecules bound to its surface at defined locations or localized areas. The molecules bound to the carrier comprise nucleic acid sequences, the capture molecules, which are specific for a given or desired target sequence. The sequences may be bound to the carrier via spacer molecules, which bind each capture nucleotide to the surface of the support. In the above context a localized area is an area of the carrier's surface, which contains capture molecules, preferably attached by means of spacers to the surface of the carrier, and which capture molecules are specific for a determined target/probe molecule.

“Spacers” are molecules that are characterized in that they have a first end attached to the biological material and a second end attached to the solid carrier. Thus, the spacer molecule separates the solid carrier and the biological material, but is attached to both. The spacers may be synthesized directly on or may be attached as a whole to the solid carrier at the specific locations, whereby masks may be used at each step of the process. The synthesis comprises the addition of a new nucleotide on an elongating nucleic acid in order to obtain a desired sequence at a desired location by for example photolithographic technologies which are well known to the skilled person. Bindings within the spacer may include carbon-carbon single bonds, carbon-carbon double bonds, carbon-nitrogen single bonds, or carbon-oxygen single bonds. The spacer may be also designed to minimize template independent noise, which is the result of signal detection independent (in the absence) of the template. In addition, the spacer may have side chains or other substitutions. The active group may be reacted by suitable means to form for example preferably a covalent bound between the spacer and solid carrier, capture or probe molecule. Suitable means comprise for example light. The reactive group may be optionally masked/protected initially by protecting groups. Among a wide variety of protecting groups, which are useful are for example FMOC, BOC, t-butyl esters, t-butyl ethers. The reactive group is used to build to attach specifically thereto (after the cleavage of the protecting group) another molecule.

The “localized area” is either known/defined by the construction of the micro-array or is defined during or after the detection and results in a specific pattern. A spot is the area where specific target molecules are fixed on their capture molecules and approved by a detector.

As used herein, the term “carrier” or “support” refers to any material that provides a solid or semi-solid structure and a surface allowing attachment of molecules. Such materials are preferably solid and include for example metal, glass, plastic, silicon, and ceramics as well as textured and porous materials. They may also include soft materials for example gels, rubbers, polymers, and other non-rigid materials. Preferred solid carriers are nylon membranes, epoxy-glass and borofluorate-glass. Solid carriers need not be flat and may include any type of shape including spherical shapes (e.g., beads or microspheres). Preferably solid carriers have a flat surface as for example in slides (such as object slides) and micro-titer plates, wherein a micro-titre plate is a dished container having at least two wells.

The expression “attached” describes a non-random chemical or physical interaction by which a connection between two molecules is obtained. The attachment may be obtained by means of a covalent bond. However, the attachments need not be covalent or permanent. Other kinds of attachment include for example the formation of metalorganic and ionic bonds, binding based on van der Waal's forces, or any kind of enzyme substrate interactions or the so called affinity binding. An attachment to the surface of a carrier or carrier may be also referred to as immobilization.

A “determinant” relates to a factor responsible for a resistance in S. aureus, which may be acquired by the micro-organism via horizontal gene transfer and which actively counteracts the effect of an antibiotic. Particularly, genetic factors, such as the mecA, aacA-aphD, tetK, tetM, vat(A), vat(B), vat(C), erm(A), erm(C) genes, which may be present on plasmid(s) or also may be incorporated in the genome of S. aureus, are envisaged.

The term “resistance mutation” as used herein refers in its widest sense to a trait of S. aureus endogenously developed, by e.g. a mutation of a protein, representing the target of the antibiotic, so that the antibiotic is not as effective any more. A resistance mutation may have the form of single nucleotide polymorphism in a gene or a target polypeptide, which applies in the case of the development of resistance to quinolones in the gene for the α-subunit of the DNA topoisomerase (in that case grlA, grlB, gyrA and gyrB).

The terms “complementary” or “complementarity” are used in reference to polynucleotides (i.e., a sequence of nucleotides such as an oligonucleotide or a target nucleic acid) in the light of the base-pairing rules. Complementarity may be partial, in which only some bases of the nucleic acids are matched according to the base pairing rules. Alternatively, there may be a complete complementarity between the nucleic acids in such a way that there are no mismatches. The degree of complementarity between nucleic acid strands has significant effects on the stringency and strength of the hybridization between two different nucleic acid strands. Complementarity as used herein is not limited to the predominant natural base pairs. Rather, the term also encompasses alternative, modified and non-natural bases, including but not limited to those that pair with modified or alternative patterns of hydrogen. With regard to complementarity, it is important for some applications to determine whether the hybridization represents a complete or partial complementarity. If it is desired for example to detect the presence or absence of a particular DNA (such as from a virus, bacterium, fungi or protozoan), the only important condition is that the hybridization method ensures hybridization when the relevant sequence is present. Other applications in contrast, may require that the hybridization method distinguish between partial and complete complementarity, for example in the detection of genetic polymorphisms.

The term “homology” and “homologous” refers to a degree of identity. There may be partial homology or complete homology. A partially homologous sequence is one that is less than 100% identical to another sequence.

“Hybridization” is used in reference to the pairing of complementary nucleic acids. Hybridization and the strength of hybridization (i.e., the strength of the association between the nucleic acids) is influenced by such factors as the degree of complementarity between the nucleic acids, stringency of the conditions involved, and the melting temperature of the formed hybrid. Hybridization involves the annealing of one nucleic acid to another complementary nucleic acid, i.e., a nucleic acid having a complementary nucleotide sequence.

“Stringency” refers to the conditions, which are involved in a correct hybridization event, for example temperature, ionic strength, pH and the presence of other compounds, under which nucleic acid hybridizations are conducted. Under conditions of high stringency, nucleic acid base pairing will occur only between nucleic acid fragments that have a high frequency of complementary base sequences. Thus, conditions of weak or low stringency are often required when it is desired that nucleic acids that are not completely complementary to one another be hybridized or annealed together.

A “marker” or “label” refers to any atom or molecule that may be used to provide a detectable (preferably quantifiable) effect and that can be attached to a nucleic acid. Markers may include colored dyes; radioactive labels; binding moieties such as biotin; haptens such as digoxgenin; luminogenic, phosphorescent or fluorogenic moieties; and fluorescent dyes alone or in combinatiori with moieties that can suppress or shift emission spectra by the energy transfer of fluorescence. Markers may provide signals, which are detectable for example by fluorescence, radioactivity, colorimetry, gravimetry, X-ray diffraction or absorption, magnetism and enzymatic activity. A marker may be a charged moiety (positive or negative charge) or may also have a neutral charge. They may include or consist of nucleic acid or protein sequence. Preferred markers are fluorescent dyes.

A “target” or “probe molecule” refers to a nucleic acid molecule to be detected. Target nucleic acids may contain a sequence that has at least a partial complementarity with at least a probe oligonucleotide.

“Probes” or “probe molecules” refer to nucleic acids, which interact with/hybridize to a target nucleic acid to form a detection complex.

The term “signal probe” or “probe” relates to a probe molecule, which contains a detectable moiety, which are already outlined above.

The term “nucleic acid” is meant to comprise any sequence of deoxyribonucleotides, ribonucleotides, peptido-nucleotides, including natural and/or artificial nucleotides.

The expression “sample” is meant to include any specimen or culture of biological and environmental samples or nucleic acid isolated therefrom. Biological samples may be animal, including human, fluid, such as blood or urine, solid or tissue, alternatively food and feed products and ingredients such as dairy items, vegetables, meat and meat by-products. Environmental samples include environmental material such as surface matter, soil, water, industrial samples and waste, for example samples obtained from sewage plant, as well as samples obtained from food and dairy processing instruments, apparatus, equipment, utensils, disposable and non-disposable items. The sample may be used as such in the assay or may be subjected to a preliminary selection step, such as e.g. culturing the sample under conditions favoring or selecting for S. aureus in said sample. Also, the nucleic acids contained in the sample may be isolated prior to performing the assay. In the presence of a multi-resistant S. aureus in the sample the resulting nucleic acid sample will contain the target nucleic acid which may be isolated from the biological sample in any way known to the skilled person, including conventional isolation comprising lysis of the cellular material of the biological sample and isolation of DNA or RNA therefrom. In case the target nucleic acid is present in a low amount, the said nucleic acid may be subjected to PCR, to specifically amplify the target nucleic acid prior to performing the assay.

A “nucleic acid sample” may be a polynucleotide or oligonucleotide of a variable length and is represented by a molecule comprising at least 5 or more deoxyribonucleotides, preferably about 10 to 1000 nucleotides, more preferably about 20 to 800 nucleotides and more preferably about 20 to 100 or even more preferred about 20 to 60. The exact size will depend on many factors, which in turn depend on the ultimate function or use of the oligonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another.

Additional features and advantages of the present invention are described in, and will be apparent from, the following Detailed Description of the Invention.

### DETAILED DESCRIPTION OF THE INVENTION

According to an embodiment, the present DNA micro-array comprises a carrier or support on which in the form of a specific pattern, nucleic acids for targeting at least 5 determinants and a resistance mutation of S. aureus are immobilized. For a correct determination of the presence of multi-resistant S. aureus in a sample a number of at least five determinants and a resistance mutation have proven to yield a doubtless, non-ambiguous result. Since all of the known nine resistant determinants offer an equal significance, the five determinants may be randomly selected from the group consisting of sequences as identified by Seq. ID. No. 1 to Seq. ID. No. 9., i.e. without any requirements concerning the selection. Preferably, the DNA micro-array comprises 6 determinants, more preferably 7 determinants, still more preferably 8 determinants and most preferably 9 determinants and thus comprises the all of the Seq. ID. No. 1 to Seq. ID. No. 9.

The nucleic acids for targeting the resistance mutation of S. aureus may comprise any sequence derived from a S. aureus gene, that conveys resistance to an antibiotic. According to a preferred embodiment, the said nucleic acid comprises a sequence derived from the gene encoding the α-subunit of the DNA-topoisomerase grIA of S. aureus, more preferably a sequence comprising the sequence as identified by Seq. ID. No. 10, wherein position no. 9 in said sequence exhibits all of the different nucleotides, i.e. A, C, T and G. That is the “localized area” containing the nucleic acids for targeting the resistance mutation may contain a nucleic acids comprising 4 different sequences (i.e. Seq. ID. No. 10 with 4 times different nucleotides at position 9), or alternatively 4 localized areas may be provided for targeting the resistance mutation, wherein each localized area comprises 1 distinct sequence of SEQ Id. No. 10.

The micro-array may also include specific controls. These controls may be embodied by including sequences in the micro-array as identified by any of Seq. ID. No. 11 to Seq. ID. No. 15 (cf. tab. 1). The controls comprise positive (e.g. a nucleic acid sequence derived from the 16 S RNA of an ubiquitous S. staphylococcus) and negative controls (e.g. nucleic acid sequences derived from different micro-organisms) and are intended to provide a control of the hybridization efficiency of the sample nucleic acids to the immobilized nucleic acids/capture probes. The controls may also comprise a spotting control, that inherently harbors a fluorescent label (e.g. NH₂-mecA-F), which may be used to check the performance of the spotting process and to facilitate orientation on the array.

The carrier or support of the present DNA micro-array may consist of different materials, preferably of glass, silicon, silica, metal, plastics or mixtures thereof prepared in format selected from the group of slides, discs, gel layers and/or beads. The carrier may also be a microplate or a slide and may consist of epoxy glass. A preferred support is for example an epoxy modified glass slide purchased by Elipsa A G, Berlin, Germany.

Preferably, the micro-array has at least 100 molecules per square centimeter attached to the solid carrier. This density may, however, be higher and be adapted to the respective application of the micro-array, in that also other suitable applications, e.g. for the determination of resistances in other organisms different from S. aureus, may be performed. For example, the density of the nucleic acids attached per square centimeter of solid carrier amounts more preferably at least to 1.000, still more preferably at least to 5.000 and most preferably at least to 10.000 nucleotides per square centimeter.

Said specific pattern allows the mapping of each nucleic acid to a specific position on said carrier and a specific analysis, in that the analysis of the results of the present DNA micro-array is facilitated and non-ambiguous concerning the attribution of a particular spot to a previous attached nucleic acid probe.

According to another preferred embodiment the present invention also provides a method for the detection of the presence of a multi-resistant S. aureus in a sample material, by determining determinants and a resistance mutation of S. aureus using a DNA micro-array.

The method comprises a step to obtain a sample material of interest. Prior to performing the method of the present invention the sample may be pre-treated e.g. centrifuging or filtering to separate non-soluble matter or selecting for S. aureus in the sample. This may be achieved by e.g. culturing the sample under conditions favouring the growth of S. aureus. Also, to improve performance, nucleic acids contained in the sample material may be isolated and/or amplified. The sample and/or the isolated/purified nucleic acid material is applied to the surface of the present micro-array. Said sample is now allowed to hybridize to the immobilized nucleic acids, the capture probes, for targeting at least 5 determinants and a resistance mutation of S. aureus, wherein the at least 5 determinants are selected from the group consisting of the Seq. ID. No. 1 to Seq. ID. No. 9. By choosing suitable hybridisation conditions known to the skilled person, such as e.g. applying a certain stringency during hybridization and washing, only those nucleic acids will hybridize to the immobilized nucleic acids and/or remain bound during washing steps, which exhibit a high homology to the immobilized nucleic acids. The method further comprises detecting any hybridisation events, which will be indicative of the presence of a multi-resistant S. aureus.

The nucleic acids for targeting the resistance mutation of S. aureus preferably have a sequences as identified by Seq. ID. No. 10, comprising four different sequences with one mutation at a particular location (all four nucleic acids).

The micro-array may also include specific controls. These controls may be embodied by including sequences in the micro-array as identified by any of Seq. ID. No. 11 to Seq. ID. No. 15 (cf. tab. 1). The controls comprise positive (e.g. a nucleic acid sequence derived from the 16 S RNA of an ubiquitous S. staphylococcus) and negative controls (e.g. nucleic acid sequences derived from different micro-organisms) and are intended to provide a control of the hybridization efficiency of the sample nucleic acids to the immobilized nucleic acids/capture probes. The controls may also comprise a spotting control, that inherently harbors a fluorescent label (e.g. NH₂-mecA-F), which may be used to check the performance of the spotting process and to facilitate orientation on the array.

The nucleic acid sample to be used for hybridizing to the immobilized nucleic acids consists preferably of oligonucleotides and/or polynucleotides of a length between 10 and 1000 nucleotides each, preferably shorter oligonucleotides/polynucleotides exhibiting a length of about 10 to 100 or between 20 to 60. The length may be obtained for example by the digestion of plasmid or genomic DNA with DNAse or preferably restrictions enzymes and facilitates the hybridisation.

The nucleic acid sample, which comprises oligonucleotides and/or polynucleotides, is preferably isolated from body tissues or fluids, particularly blood, suspected to contain S. aureus. Such techniques are well known to the skilled person and may be also performed with commercial available kits.

The capture and the target nucleic acids may be present in a labeled form. The target nucleic acids may be labeled prior to performing the assay, by including a marker molecule into the molecule, e.g. during its amplification or isolation. Said marker molecule is preferably a fluorescent marker. Also the capture molecules may be labeled, in case of a fluorescent dye preferably with a dye exhibiting a different excitation and/or emittance wavelength, which allows a normalization of the experiment.

Methods for the detection of binding include e.g. surface plasmon resonance or detection of fluorescence at a localized area indicative of binding of a labelled molecule. Fluorescence may be detected e.g. via confocal laser induced fluorescence.

In another embodiment, a kit is provided for the detection of S. aureus infections. Said kit either provides nucleic acids for targeting at determinants and a resistance mutation of S. aureus, as represented by nucleic acids as identified by Seq. ID. No. 1 to Seq. ID. No. 10, and optionally controls having sequences as identified by Seq. ID. No. 11 to Seq. ID. No. 15. Alternatively the kit may also provide a micro-array as detailed above.

A typical automated processing of a micro-array according to a preferred embodiment of the present invention includes the use of three components. First, the micro-array or support respectively, second a reader unit and third means for the evaluation of the results, e.g. a suitable computer software. The reader unit comprises in general a movable tray, focussing lens(es), mirrors and a suitable detector, e.g. a CCD camera. The moveable tray carries the micro-array and may be moved to place the micro-array within the light path of one or more suitable light sources, e.g. a laser with an appropriate wavelength to excite a fluorescent compound. The evaluation program or software may serve for example to recognize specific patterns on the array or to analyse different expression profiles of genes. In this case, the software searches colored points on the array and compares the intensity of different color spectra of the same point. The result may be interpreted by an analyzing unit and afterwards stored in a suitable file format for further processing.

As detailed above, the probe- and/or target-nucleic acids may be labelled each with a fluorescent dye and the intensity of the fluorescence at different wavelengths of each point is compared to the background. The detector, e.g. a photomultiplier or CCD array, transforms low light intensities to an amplifiable electrical signal. Other methods use different enzymes, which are covalently bound to the nucleotide by means of a linker molecule. The enzymatic colorimetry uses for example alkaline phosphatase and horseradish peroxidase as marker. By contacting with a suitable molecule, a detectable dye may be achieved. Other chemoluminescent or fluorescent marker comprise proteins capable to emit a chemoluminescent or fluorescent signal, if irradiated with light of a discrete, specific wavelength, e.g. 488 nm for the green fluorescent protein. Radioactive markers are applied in case of low detection limits are required, but are due to their harmful properties not wide spread. Fluorescence marking is performed with nucleotides linked to a fluorescent chromophore. Combinations of nucleotides and fluorescent chromophore comprise in general Cy3 (cyanine 3)/Cy5 (cyanine 5) labelled dUTP as dye, since they may be easily incorporated, the electron migration for fluorescence may be exited by means of customary lasers and they also have distinct emission spectra.

In the hybridisation of micro-arrays essentially the conventional conditions of southern or northern hybridisations, which are well known to the skilled person are applied. The steps may comprise a pre-hybridisation, the intrinsic hybridisation and a washing step after hybridisation occurred. The conditions have to be chosen such that background signals are kept low, minimal cross-hybridisation (in general a reduced number of mismatches) occurs and with a sufficient signal strength, which has to be proportional for some applications to the concentration of the target molecule.

The hybridisation event may be detected in any conventional way, in an automated system generally by two different kinds of array-scanners. One method employs the principle of the confocal laser microscopy, which uses at least one laser to scan the array in point-to-point manner. Fluorescence is then detected by photomultipliers, which amplify the emitted light. The less expensive GGD based readers typically use filtered white light for excitation. The surface of the array is scanned with this method in sections, which allows the faster achievement of results of a lower significance.

Also the so-called gridding for the analysis of the results may be applied, in which an idealised model of the layout of the micro-array is compared with the scanned data to facilitate spot definition. Pixels are classified (segmented) as spot (foreground) or background to produce the spotting mask. Segmentation techniques may be divided in fixed segmentation circle, adaptive circle segmentation, adaptive shape segmentation and histogram segmentation. The use of these techniques depends from the shape of the spots (regular, irregular) and the quality of the proximal arrangement of the spots.

Another issue for the evaluation of the results is the intensity of the distinct spots, since the concentration of hybridised nucleotides in one spot is proportional to the total fluorescence of this spot. In particular, the overall pixel intensity and the ratio of the different fluorescent chromophores used (in case of Cy3 and Cy5, green and red) are important for the calculation of the spot intensity. Beneath the spot intensity, also the background intensity has to be taken into account, since various effects may disturb the fluorescence of the spots, for example the fluorescence of the support and of the chemicals used for the hybridisation. This may be performed by the so-called normalisation, which includes the above-mentioned effects and others like fluctuations of the light source, the lower availability/incorporation of the distinct marker molecules (Cy5 worse than Cy3) and their differences in emission intensities. Of importance for the normalisation is further the reference against which shall be normalized. In general, this may be a specific set of genes or a group of control molecules present on the micro-array.

The results may be further processed by means of the available software tools and according to the knowledge of bioinformatics.

It is to be understood that the above description is intended to be illustrative only and not restrictive. Many embodiments will be apparent to those of skill in the art upon reviewing the above description. By way of example, the invention has been described preliminary with reference to the use of nucleic acids for the resistance determinants and a resistance mutation of S. aureus in present method, kit and DNA micro-array. It should be clear that also other resistance determinants may be selected, dependent on the genetic development of multi-resistant S. aureus strains. Also, other resistance mutation of S. aureus may applied. The scope of the invention should, therefore, be determined not with reference to the above description, but should instead be determined with reference to the appended claims, along with the full scope of equivalents to which such claims are entitled.

### EXAMPLES

A. Bacterial Strains and DNA Extraction

S. aureus isolates investigated in this study originated from material obtained from the National Reference Center for Staphylococci in Germany. To evaluate oligonucleotide capture probes for the detection of various resistance genes, the following, previously characterized strains were used: the multi-resistant isolate S. aureus 694/01 [the reference strain for mecA, aacA-aphD, tetK, tetM, erm(A) and erm(C)] was taken from the in house strain collection. S. aureus ES 1767 [the reference strain for vat(A)], ES 1768 [vat(B)] and ES 1877 [vat(C)] were kindly provided by N. El Solh, Paris, France. All strains were grown on sheep blood agar. Staphylococcal genomic DNA was extracted from 2 ml overnight culture with the DNeasy Tissue Kit (Qiagen, Hilden, Germany) following the manufacturer's instructions and using lysostaphin (100 μg/ml, Sigma, Taufkirchen, Germany) to achieve bacterial lysis.

B. Antimicrobial Susceptibility Testing

All isolates were tested by the broth microdilution assay as described in the NCCLS standard (National Committee for Clinical Laboratory Standards. 2001. Methods for dilution antimicrobial susceptibility tests for bacteria that grow aerobically. Approved standard M7-A4, In National Committee for Clinical Laboratory Standards, Wayne, Pa.), except that Iso-Sensitest broth (Oxoid, Wesel, Germany) was used.

C. Primers and Probes

The primers used to amplify the different loci in a multiplex PCR approach are described in tab. 1. For the amplification of the relevant fragment of the DNA topoisomerase gene the following primers were used:

gr1Af - 5′-GTG CAT TGC CAG ATG TTC GTG AT-3′
and
gr1Ar - 5′GCT TAA CTT AGC TTC AGT GTA-3′

Primers and probes were selected from public databases using the software Primer3 freely available via the internet (http://frodo.wi.mit.edu/cgi-bin/primer3/primer3_www.cgi), and synthesized by Metabion (Munich, Germany). Oligonucleotide capture probes were synthesized with a 5′-terminal amino-modification for covalent coupling to the slide surface and a 10 residues T spacer to improve hybridization efficiency. All probes were designed in such a way that they exhibit similar melting temperatures (cf. tab. 1) to facilitate uniform hybridization conditions and to prevent high divergence in signal intensities. The specificity of the probes was verified in a BLAST search available through the National Center for Biotechnology Information website (www.ncbi.nlm.nih.gov).

D. Controls

In addition to the amplicon specific capture probes several control probes were designed. A fluorescein labeled spotting control (Seq. ID. No. 15) was used to check the spotting quality and to facilitate orientation on the array; negative and positive hybridization controls (Seq. ID. No. 12 and Seq. ID. No. 13, respectively) were selected to control the hybridization step; the latter one was complementary to a fluorescein labeled oligonucleotide (Seq. ID. No. 14), which was spiked during the hybridization step; a process control (Seq. ID. No. 11), targeting the PCR amplification control, monitored the efficiency of PCR amplification, labeling and hybridization, and was used for signal normalization in the data evaluation step.

E. Oligonucleotide Array Fabrication

Lyophilized oligonucleotide probes (HPLC purity grade) were dissolved in spotting buffer (160 mM Na₂SO₄, 130 mM Na₂HPO₄) to a final concentration of 20 μM and spotted using a MicroGrid II equipped with MicroSpot 2500 pins (BioRobotics, Cambridge, UK) on epoxy modified glass slides (Elipsa AG, Berlin, Germany). For covalent immobilization of the oligonucleotides the array was incubated at 120° C. for 30 minutes. All capture probes were spotted in triplicate with the resulting spots having an average size of around 150 μm. Prior to hybridization, slides were blocked; therefore they were rinsed for 5 minutes in washing solution I (0.1% (v/v) Triton X 100), for 4 minutes in washing solution 11 (0.5 μl conc. HCl per ml aqua bidest.) and for 10 minutes in washing solution III (100 mM KCl) while constantly stirring. Subsequently, the slides were incubated, with the spotted side upwards, in blocking solution (25% (v/v) ethylenglycol, 0,5 μl conc. HCl per ml a. bidest.) for 20 minutes at 50° C. Finally they were rinsed in a. bidest for 1 minute and dried by centrifugation.

F. PCR Amplification and Labeling

Single PCR products generated from genotypically characterized reference strains using PCR beads (Amersham Biosciences, Freiburg, Germany) were used to select appropriate capture probes. To characterize a selection of clinical isolates, a multiplex PCR amplification strategy as described previously has been chosen (Strommenger, B. et al; J. Clin. Microbiol. 41 (2003), 4089-4094). Routinely, 0.25 μl (approximately 10 ng) template DNA in a 25 μl volume were used to amplify fragments of 9 different antibiotic resistance genes and a fragment of the staphylococcal 16S rDNA as internal control. In order to determine the detection limit of micro-array based resistance gene detection, various amounts of template DNA (10 pg, 100 pg, 1 ng, 10 ng) were used in the PCR reaction. PCR products were purified using the QIAquick PCR purification kit (Qiagen, Hilden, Germany). To compare the results of PCR and micro-array hybridization respectively, PCR products (1 μl) were separated using the Agilent 2100 Bioanalyzer together with the DNA 1000 LabChip kit (Agilent Technologies, Boblingen, Germany). 16 μl of the purified PCR products were fluorescein labeled in a random primed labeling reaction with Fluorescein HighPrime (Roche, Mannheim, Germany) according to the manufacturer's instructions. Alternatively, a photochemical labeling of PCR products with Psoralen-PEO-Biotin (Pierce Chemicals, Rockford, USA) was used, in which the same amount of PCR product was labeled in a 20 μl reaction volume containing a final concentration of 200 μM Psoralen-PEO-Biotin. Photoreactive labeling occurred during a 30 minute exposure to long UV-light (365 nm). 20 μl of labeled multiplex PCR product (the whole labeling reaction mixture) was hybridized to the array without further purification. For combined hybridization of multiplex PCR products and the grlA amplicon, PCR products were purified and labeled as described above before they were pooled for hybridization.

The signal intensities obtained using Psoralen-PEO-Biotin in combination with Streptavidin-Cy3 conjugate with this approach were higher. However, variation in signal intensities between different capture probes was reduced but still apparent. Due to the modified intensity values the thresholds of the evaluation concept to the following were adapted: mean process control >25.000, relative signal intensity for positive capture probes >0.25.

G. Array Hybridization and Washing

Hybridization of denatured labeled PCR products was performed in 130 μl of 3×SSPE using doubled Gene Frames and appropriate cover slips (Thermo Life Science, Dreieich, Germany) in an Eppendorf thermomixer equipped with an exchangeable slides thermoblock (Eppendorf, Hamburg, Germany) for 4 hours at 42° C. with agitation (1200 rpm). To control hybridization efficiency, the hybridization mixture contained 0.25 μl of a 5′-terminal fluorescently labeled oligonucleotide complementary to the hybridization control capture probe (Seq. ID. No. 14, 0,05 μM). After hybridization the slides were washed with 2×SSC, 0.5% SDS, then with 1×SSC and finally with 0.1×SSC, each time for 10 minutes at room temperature, before they were dried by centrifugation. In case of Psoralen labeling the array was incubated with 15 μl Streptavidin-Cy3 conjugate (Amersham Biosciences, Freiburg, Germany), diluted 1:500 in TBST buffer, for 15 minutes under a glass coverslip.

H. Data Acquisition and Processing

Fluorescent images of the micro-arrays were obtained by scanning the slides with an ArrayWorX biochip reader (Applied Precision, Inc., Marlborough, UK) using a resolution of 9.750 μm and the 530 nm and 590 nm filter, respectively. Fluorescence signal intensities from each spot as well as the intensity values for the local background were analyzed by use of the ArrayWorX software. The resulting raw data was further processed using Excel (Microsoft). For calculation of individual net signal intensities (herein referred to as signal intensity, SI) the local background was subtracted from the corresponding raw spot intensity values. A mean intensity value for each capture probe was assessed from the three replicate spots for each probe. That mean intensity value was normalized to the mean intensity value of the process control probes (the resulting value is herein referred to as relative signal intensity).

For the detection of SNPs in grlA an alternative “internal” normalization strategy according to Grimm et al.; J. Clin. Microbiol. 42 (2004) pp. 3766-3774 was chosen. Within the probe set the probe with the highest mean signal intensity was considered the perfect match (PM), the remaining three probes were considered mismatches (MM). For comparison “internal” relative signal intensities were calculated by normalizing the mean signal intensities of all SNP probes to that of the PM probe resulting in relative intensities of 1 for all PM probes and relative intensities below 1 for all MM probes.

I. Detection Limit

To assess the detection limit of the presented micro-array system repeated experiments with descending amounts of DNA (10 ng to 10 pg) from the genotypically characterized strain 694/01 (data not shown) were conducted. Reliable results were obtained from a minimum amount of 100 pg to 1 ng bacterial DNA. Below that, signal intensities were markedly reduced and problems with wrong positive results occurred due to increasing background fluorescence. Although signal intensities for Psoralen labeling were generally higher, the detection limits were roughly the same for both labeling approaches. Variation in results between 1 ng and 100 pg were mainly attributed to differences in spotting quality of slides from different spotting charges, but influences of other factors like amplification and labeling efficiency must be taken into consideration.

J. Combined Detection of Resistance Genes and Mutations

For combined detection of resistance genes and mutations the most common mutation in grlA leading to quinolone resistance in S. aureus, S80F and S80Y respectively were detected. The probe set for this SNP detection consisted of 4 identical probes differing only at the central position covering the base of interest. To optimize this probe set with regard to signal intensity and discriminatory power, single grlA PCR products from genotypically defined strains were hybridized. The optimized probe set (tab. 1) was integrated into the array and single PCR products from strains sensitive and resistant to ciprofloxacin were hybridized. All array results were controlled by sequencing of the PCR product and results corresponded to the results of phenotypic antibiotic resistance testing (tab. 2). Since signal intensities for the grlA probe set were comparatively low, Psoralen labeling turned out to be superior to the HighPrime labeling approach. The parallel detection of resistance genes and the determination of the grlA allele worked reliable in repeated experiments. However, the data evaluation were separated for two reasons. (i) The low mean signal intensity for the grlA probe set, which was attributed to their reduced length necessary for a reliable discrimination between the two respective alleles, in several hybridization experiments led to relative signal intensities below the threshold for positive hybridization reactions. (ii) Although discrimination between the four different alleles was good, the three mismatch probes showed significant background fluorescence, especially if the allele “C” was detected as perfect match; thus after signal normalization to the independent process control (which showed multiple mean signal intensity) discrimination between perfect match and mismatch was hampered. Using “internal” normalization to the perfect match probe relative intensity values for mismatch probes remained below 0.4 in all hybridization experiments conducted, indicating the high discriminatory power and diagnostic reliability of the system.

K. Testing of Clinical Isolates and Correlation to Phenotypic Antibiotic Resistance Testing

13 different clinical isolates were tested with the present DNA micro-array. The results of the DNA micro-array experiments were compared with those obtained by PCR and phenotypical resistance testing, respectively. Hybridization experiments were conducted repeatedly, using either labeling methods. Micro-array results obtained from both methods were identical and are summarized in table 2. They were further confirmed by PCR detection of each of the resistance determinants and were concordant with results of the phenotypic antibiotic susceptibility testing.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A micro-array comprising a carrier and immobilized thereon in the form of a specific pattern nucleic acids comprising sequences specific for at least 5 determinants and a nucleic acid comprising a sequence, specific for a resistance mutation of Staphylococcus aureus, wherein said nucleic acids specific for the at least 5 determinants are selected from the group consisting of the Seq. ID. No. 1 to Seq. ID. No. 9.

2. The micro-array according to claim 1, wherein said nucleic acid sequence specific for the resistance mutation of Staphylococcus aureus has a sequence as identified by Seq. ID. No. 10.

3. The micro-array according to claim 1, wherein the DNA micro-array also includes controls selected from the group of sequences as identified by Seq. ID. No. 11 to Seq. ID. No. 15.

4. The micro-array according to claim 1, wherein said carrier consist of a material selected from the group consisting of glass, metal and plastic.

5. The micro-array according to claim 4, wherein said carrier consists of epoxy glass.

6. The micro-array according to claim 4, wherein said carrier is selected from the group consisting of a micro-plate and a slide.

7. The micro-array according to claim 1, wherein the surface of said carrier comprises an area of at least 1 square centimetre.

8. The micro-array according to claim 1, wherein said nucleic acids specific for at least 5 determinants and a resistance mutation of Staphylococcus aureus are attached to the surface of said carrier with a density of at least 100 molecules per square centimetre.

9. The micro-array according to claim 1, wherein said specific pattern allows mapping of each nucleic acid to a specific position on said carrier and a specific analysis.

10. The micro-array according to claim 1, wherein said nucleic acids are bound to the carrier via a spacer molecule.

11. A method for the detection of multi-resistant strains of S. aureus, comprising the steps of
a) providing a DNA micro-array comprising a carrier and immobilized thereon in the form of a specific pattern nucleic acids comprising sequences specific for at least 5 determinants and a sequence, specific for a resistance mutation of Staphylococcus aureus, wherein said nucleic acids for targeting at least 5 determinants are randomly selected from the group consisting of the Seq. ID. No. 1 to Seq. ID. No. 9.
b) contacting a biological sample with said micro-array under conditions allowing hybridization; and
c) detecting at least one hybridization event;
wherein a hybridization event to the sequence, specific for a resistance mutation and to at least one sequence specific for a determinant, is indicative of the presence of a multi-resistant S. aureus strain in said sample.

12. The method according to claim 11, wherein said nucleic acid specific for the resistance mutation of Staphylococcus aureus has a sequence as identified by Seq. ID. No. 10.

13. The method according to claim 11, wherein the DNA micro-array also includes specific controls selected from the group of sequences as identified by Seq. ID. No. 11 to Seq. ID. No. 15.

14. The method according to claim 11, wherein said sample comprises target oligonucleotides and/or polynucleotides, exhibiting a length of about 10 to 100 nucleotides.

15. The method according to claim 14, wherein said oligonucleotides and/or polynucleotides are isolated from body tissues or fluids suspected to contain Staphylococcus aureus.

16. The method according to claim 11, wherein said target nucleic acids are labelled with a marker molecule.

17. The method according to claim 16, wherein said marker molecule is selected from the group consisting of cyanine dyes, renaissance dyes, and fluorescent dyes.

18. A diagnostic kit for the detection of Staphylococcus aureus infections, comprising nucleic acids for targeting at determinants and a resistance mutation of Staphylococcus aureus, consisting of the Seq. ID. No. 1 to Seq. ID. No. 10 and/or a micro-array according to a micro-array comprising a carrier and immobilized thereon in the form of a specific pattern nucleic acids comprising sequences specific for at least 5 determinants and a nucleic acid comprising a sequence, specific for a resistance mutation of Staphylococcus aureus, wherein said nucleic acids specific for the at least 5 determinants are selected from the group consisting of the Seq. ID. No. 1 to Seq. ID. No. 9.

19. The method according to claim 14, wherein said oligonucleotides and/or polynucleotides are isolated from blood suspected to contain Staphylococcus aureus.

20. The method according to claim 16, wherein said marker molecule is selected from the group consisting of cyanine dyes comprising Cy3 and Cy5.

21. The method according to claim 16, wherein said marker molecule is selected from the group consisting of renaissance dyes comprising ROX and R110.

22. The method according to claim 16, wherein said marker molecule is selected from the group consisting of fluorescent dyes, preferably FAM and/or FITC.

23. The diagnostic kit for the detection of Staphylococcus aureus infections of claim 18, comprising the controls having a sequence selected from the group consisting of the Seq. ID. No. 11 to Seq. ID. No. 15.
